(19) United States
(12) Patent Application Publication
(10) Pub. No.: US 2013/0304004 A1
(43) Pub. Date: Nov. 14, 2013

(54) COLLECTION DEVICE FOR USE IN A DRAINAGE DEVICE

(71) Applicant: Birgit Riesinger, Munster (DE)

(72) Inventor: Birgit Riesinger, Munster (DE)

(21) Appl. No.: 13/866,895

(22) Filed: Apr. 19, 2013

Related U.S. Application Data
(63) Continuation of application No. PCT/EP2011/068357, filed on Oct. 20, 2011.

(30) Foreign Application Priority Data
Oct. 20, 2010 (DE) 10 2010 060 073.3

(51) Int. Cl. A61M 1/00

(52) U.S. Cl. CPC A61M 1/0001
USPC 604/319

(57) ABSTRACT

What is provided is a collection device for use in a drainage device for treating wounds using negative pressure, wherein said collection device is provided entirely or partially with at least one insert containing liquid-absorbing polymers.

### CROSS-REFERENCE TO RELATED APPLICATIONS

The present application claims priority from and is a continuation from PCT Application Serial No. PCT/EP2011/068357, filed Oct. 20, 2011, which claims priority from German Patent Application Serial No. DE 10 2010 060 073.3, filed Oct. 20, 2010, all of which are incorporated by reference in their entireties.

### BACKGROUND

The invention relates to a collection device for use in a drainage device for negative pressure wound therapy.

In treatment of open wounds, sutures and staples are the current procedures. However, such mechanical closing procedures have a drawback in that they exert great tension on the tissue around the wound, and under certain circumstances can even result in ruptures. In addition, these procedures cannot be used with severely inflamed wounds and/or deep open wounds.

For treatment of such wounds, drainage procedures have long been employed, in which, through application of continuous negative pressure on the wound, the wound can be kept dry and clean, so that migration of epithelial tissue and subcutaneous tissue into the wound is improved. Wound exudate is collected in a collection container such as a vacuum bottle and then disposed of. However, the collection containers used in current drainage procedures for liquid wound exudates have significant drawbacks. One of the main problems with them is correctly and safely dealing with fluids that in part are highly infectious. These can, for example, spill during changing of the collection device.

WO96/05873 describes a wound drainage system in which the collection container is additionally filled with a gel-forming substance such as polyacrylamide, so that the drainage fluid collected therein is immobilized. Through the binding of the wound exudate, the danger of contamination is lessened, since as a rule even if the collection device is tipped, no fluid can escape. However, the loose filler of such a gel-forming substance has a drawback in that it is not possible to do a check via a specific expansion of the substance in a bubbled-up state. It is true that the liquid is very largely immobilized, but the mass as such remains portable within the collection device, and therefore can continue to result in contaminations by spills of the bubbled mass. In addition, such containers remain hard to clean and sterilize, if the gel is not disposed of together with the container.

### SUMMARY OF THE INVENTION

The object of the present invention, among other things, is, to better manage the exudate obtained during negative pressure wound therapy, in regard to the contamination risk proceeding from it and in regard to the physiological and pathological significance inherent in it. This problem is solved with the features of the claims presented.

### BRIEF DESCRIPTION OF THE DRAWINGS

In the accompanying figures, like elements are identified by like reference numerals among the several preferred embodiments of the present invention.

FIG. 1 shows a collection device 5 with a lid 30 and spray head 22 with a loose filler of liquid-absorbing substances.

FIG. 2 shows an invention-specific collection device 5 with a lid 30 and spray head 22 with an inner lining made of an immobilized, liquid-absorbing insert 16.

FIG. 3 shows an invention-specific collection device 5 with a lid 30 and spray head 22 with multiple immobilized liquid-absorbing layers 16, which are encased with foil or film 17.

FIG. 4a shows a collection device 5 with an inner container 15 and a liquid-absorbing insert 16.

FIG. 4b shows the removal of the inner container 15 from the collection device 5 with a liquid-absorbing insert 16 in a used state.

FIG. 5 shows an invention-specific collection device 5 with a lid 30 and a bottle-shaped spray head 22 with an inner lining made of an immobilized liquid-absorbing insert 16.

FIG. 6 shows an invention-specific collection device 5 with a pivoting lid 30 and a spray head 22 with an inner lining made of an immobilized liquid-absorbing insert 16.

FIG. 7a shows an invention-specific collection device 5 with a bag-shaped inner container 15.

FIG. 7b shows the removal of the bag-shaped inner container 15 from the collection device 5.

FIG. 8 shows a spiral-shaped spray head 22 with exit openings 23.

FIG. 9 shows a helical spray head 22 with exit openings 23.

FIG. 10 shows a collection device 5 with a distributor 50.

FIG. 11 shows the installed position of the collection device 5 in relation to the overall drainage device 100 depicted in FIG. 14.

FIG. 12 shows the installed position of the collection device 5 in relation to the overall drainage device 100 depicted in FIG. 14 with various supply units 31, 35, 36.

FIG. 13a shows a collection device 5 with a lid 30 and a spray head 22 integrated into the lid.

FIG. 13b shows the lid 30 with the spray head 22 and the exit openings 23 in a top-down view.

FIG. 14 shows the invention-specific device 100 in its entirety.

FIG. 15a shows a vertical section of a comminution device 48 for installation in a drainage hose

FIG. 15b shows a cross section of a comminution device 48 for installation in a drainage hose.

FIG. 15c shows the installation of a comminution device 48 into a drainage hose 4.

FIGS. 16a and 16b and 17a and 17b show additional embodiment forms of a stationary vacuum pump.

FIGS. 18-21 show additional embodiment forms of a portable vacuum pump.

### DETAILED SPECIFICATION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

According to the invention, a collection device is provided for use in a drainage device for negative pressure wound therapy, wherein the said collection device is provided in part or entirely with at least one liquid-absorbing insert containing polymers.

In an alternative embodiment form, an analysis device is provided for investigation of the body's own exudates obtained by suction for physiological and/or pathological parameters.

Preferably the collection device or the analysis device is inserted in a drainage device for negative pressure wound therapy, which additionally exhibits a gastight wound-covering dressing, a suction head, at least one drainage hose, and, optionally, a source of negative pressure.

In its simplest embodiment form, the invention-specific collection device comprises, for example, a mat which contains liquid-absorbing polymers. Such a mat can, for example, exhibit a cellulose fleece, and can be inserted with or without a sheathing into a drainage device for negative pressure wound therapy.

Alternative provision can be made that the collection device has a container which contains the said insert. Preferably the container exhibits a rigid wall, pressure-resistant if necessary. Such a container can, for example, assume the shape of a canister or a bottle, similar to a vacuum bottle.

The insert containing liquid-absorbing polymers may be a loose filler.

Alternative provision can preferably be made that the insert containing liquid-absorbing polymers is spatially defined or limited, for example in the form of a textile material, a bag, a wall coating, a single-use container, containing the said polymers or the like. The advantages of all these embodiments consist in better hygiene, better protection against spilling or running out when, for example, the device is tipped, and/or—in some embodiment forms—easier removal of the said insert. These advantages are described in particular below or are evident from the figures.

The said source of negative pressure preferably is selected from the group comprising

a) Electrically driven vacuum pumps

b) Manual vacuum pumps, and/or

c) An evacuated vacuum vessel.

The said vacuum pump may be a solitary pump, but also can be a component of a centralized suction system, as is often used in a medical clinic. In patients' rooms, wall-mounted vacuum attachments are arranged, to which the invention-specific drainage devices can be attached for wound treatment. In this case, the said vacuum pump can place a plurality of invention-specific drainage devices for negative pressure wound therapy. In principle, any suitable pump system can be used, as long as the pump is capable. The pump must be capable of pulling negative pressures of −80 to −200 mm Hg, and preferably it must also be capable of transporting fluids.

Similar to the known vacuum bottle, the said evacuated vessel can be attached to the invention-specific device for wound therapy and thus placed under negative pressure. The said evacuated vessel exhibits an insert containing a liquid-absorbing polymer, preferably in the form of a wall lining.

What is in mind here especially—and this holds true for all the embodiment forms discussed in this application—is not merely the classical vacuum wound care systems, but also post-operative drainage systems such as those used, for example, after an arthroscopy.

In especially preferred fashion, the said evacuated vessel can be provided in the form of a cartridge which is inserted in a holding device, which is already connected with the invention-specific drainage device for wound therapy. When the cartridge is full, it is removed and disposed of, and a new evacuated cartridge can be inserted into the holding device.

The previously named embodiments are especially preferred, because by dispensing with its own pump, and instead using an evacuated vessel, the device is portable and independent of a network, so that the patient himself becomes mobile. In addition, thus a small design can be achieved, which permits the patient to discretely conceal the device. For this, an anatomically adapted embodiment of the said evacuated vessel or of the holding device mentioned is advantageous, making it possible inconspicuously to carry the same on a leg, for example.

Additionally, such a device does not emit any sounds while in operation and is very easy to operate.

The same holds true for the manual source of negative pressure. Here, in the simplest case, it can be a plastic sprayer with a sufficiently large volume. Other possibilities are a pump similar to a rubber ball, a bellows, etc.

At least in sections, the drainage hose mentioned can be provided with a sheathing exhibiting superabsorbing polymers or an inner wall coating exhibiting superabsorbing polymers. Then preferably it is designed to have a coaxial dual wall, with the superabsorbing polymers able to be inserted in the intermediate space between the two hose walls. In this way, if the drainage hose leaks, the exudate can be prevented from escaping; in addition, thus a self-sealing sheathing can be formed. This embodiment form has an additional advantage in that at least in sections, it strengthens the hose walls and protects them from collapsing when negative pressure is applied.

The gas-tight wound dressing serves to close the wound so that a negative pressure can be applied. Within the wound area found beneath the wound dressing, in addition an absorbing body can be inserted that takes up and absorbs additional wound exudate. To give additional protection to the wound, in addition a supplemental liquid-permeable foil or film element is conceivable (a so-called wound spacer grid), which protects the wound from adhering with the absorbing body situated above and/or the wound dressing. Such wound systems are sufficiently known and described, for example, in DE 202005010653 U1.

The wound dressing has available at least one opening for attachment of a suction head, which serves as the attachment for a drainage hose, which in turn leads via an inlet into a collection device for wound exudate. The collection device has available at least one insert containing a liquid-absorbing polymer, which is designed to take up and absorb the wound exudate. Preferably its maximum expansion capacity in a moistened state is adapted to the inner volume of the collection device.

What is to be understood by the term “maximum expansion capacity” is the largest possible expansion of the insert as a result of absorbing wound exudate. This is adapted to the inner volume of the collection device in such a way that the container, or the drainage components to be inserted therein such as hoses or a spray head, suffer no damage at maximum expansion. Also, kinking or breakage of certain system components is allowed for in a preliminary stage when providing dimensions for the insert, so that due to expansion through absorption, the function of the drainage system is not impaired.

In this connection, the term “immobilized” means that the insert containing liquid-absorbing polymers is not present as a loose filler, but is held together in a form suitable for the particular application. In a preferred embodiment form, it can be a foam, a textile material and/or a homogeneous non-textile polymeric material, which contains liquid-absorbing polymers like superabsorber particles or fibers. The insert can also be configured as a film with a layer thickness from 1 to 3 mm, which consists, for example, of CMC, water, glycerine, and superabsorber particles. Glycerine and SAPs exhibit bacterio- and fungistatic action. However, a textile material with superabsorber particles or fibers worked in is just as possible as a foam or sponge material, with appropriately added polymers. Absorber bodies that are suited for use in the collection device are sufficiently known and are part of prior art.

The liquid-absorbing insert is situated within the collection device. For this, the container can partially (i.e., only in certain areas), or entirely be covered with such an insert, for example in the form of an inner lining. The insert is either loosely applied or attached reversibly or—with single-use products—irreversibly to the inner wall of the collection device. Also, multiple layers lying one atop the other are possible in certain areas or as a complete interior lining.

Especially in regard to the monitored absorption or swelling process within the collection device, the present device has a decisive advantage, because increased handling safety is thus attained. The liquid wound exudate is immediately taken up from the liquid-absorbing inserts, and also the swollen insert continues to be present not as a loose gel mass, which could continue to run out. Thus it is precluded that the wound exudate will spill out with the associated contamination, both in liquid and in bound form.

In addition, the device offers an advantage in that the collection device is easier to disinfect, since the liquid-absorbing insert can be disposed of as a whole together with the absorbed wound exudate, and does not have to be removed as a gel-like mass from the container. And vice versa, the inserts are easier to place into the collection device than would be the case with a loose filler made of gel-forming substances. Simple disposal also means the odor is minimized, since the bound wound exudate causes no annoying odor both when in use and when disposed of. If necessary, the collection device can, if necessary, even be used multiple times with the same patient before it has to be emptied and then disinfected if necessary. A further advantage during use is that sounds annoying to the patient are reduced or eliminated, since the liquids are directly absorbed and thus the draining wound exudate makes no noise, or barely any, when it runs into the collecting device.

Additionally, various safety elements, such as tipping sensors that are to prevent a spilling of wound exudate or keep the collection device from running over, can be dispensed with, without impairing safety. Thus, the present drainage system is cheaper to design and in addition more cost-effective.

In an additional embodiment form, the liquid-absorbing insert is at least partially sheathed by a liquid-permeable foil or film, which lies flat on the insert. Due to the sheath, the insert found within it is additionally protected and held together, which is especially advantageous with flexible materials like a textile fabric or fleece with superabsorbers. The sheath tightly adjoins the absorption layer lying beneath, and can, for example, be attached on the underside to the inner wall of the collection device. A suitable example of a sheath is a foil-like or film-like material with appropriate perforations, but also a fleece material, as long as it is suited to conduct the wound exudate to the absorption body.

In a special embodiment form, between the liquid-absorbing insert and the collection device, a removable inner container, especially a bag, is placed. In essence the inner container adaptably fits into the collection device, so that the entire inner volume of the collection device is used. With a bag, the shape of the bag is preferably adapted to the collection device, so that it can be disposed of for example as a single-use product along with the absorbed wound exudate. The bag can consist of plastic or of a textile or paperlike material, similar to a vacuum cleaner bag. By this means it becomes even easier to clean and disinfect the reusable collection device. Very similar to a vacuum cleaner bag, it can also exhibit an entry valve in the form of a flexibly supported flap.

In other respects the said bag can also function as a collection device per se, i.e. in this case it is not inserted into a collection device, but rather functions itself as the said collection device.

In another embodiment form, the inner container is biodegradable. The liquid-absorbing inserts can either be pre-placed in the inner container, or inserted in the inner container after it has been placed into the collection device. As an alternative to a bag, a reusable inner container is also possible, which can be cleaned and disinfected exactly like the collection device.

As already mentioned, the inner container can consist of a single-use material, or be adapted as a solid container into the collection device and be reused. The collection device in turn may consist of glass, plastic or metal, and be designed either for reuse or as a one-time disposable product.

In another embodiment form, the liquid-absorbing insert is joined with the inner wall of the collection device or the inner wall of the inner container. It can be a reversible attachment in case the container is reused, or an irreversible attachment via adhesive bonding, for example, if this is a single-use container. If the liquid-absorbing insert is provided with a sheathing, just the sheathing can be attached to the collection device or the inner container.

In another embodiment form, spacer media are placed in the collection device, which counteract the expansion of the liquid-absorbing insert toward the middle of the container. These can, for example, be an inner basket, a (wire) wicker structure or some other type of framework which, when the liquid-absorbing insert expands, allows free space in the inner area that is not filled in by the absorption layer. At the same time, such media serve as spacers for the components which drain into the collection device. Among these, for example, is the drainage hose, measuring sensor and/or the spray head or distributor for spraying or distributing the liquid wound exudate.

This is also a feature of an additional embodiment form, in which, within the collection device, at least one spray head is arranged, attached to the inlet, with one or more exit openings. The spray head simultaneously carries out two functions: for one, by this means, a better distribution, and thus also a moistening of the liquid-absorbing inserts is assured, since the shape of the spray head and the arrangement of exit openings is adjusted as much as possible to the number and arrangement of the inserts. For another, through the fine distribution of the liquid, annoying sounds as would be the case with fluid drops, are minimized. In an ideal case, the exit openings of the spray head are directed to the liquid-absorbing inserts. The spray head, and also the collection device or the inner container, can in addition be equipped with a hydrophobic coating, to promote absorption by the liquid-absorbing inserts and to provide guidance in the appropriate direction.

In another embodiment form, the spray head is able to be pivoted and/or rotated, i.e. placed movably within the collection device. By this means the distribution of the wound exudate is improved, and at the same time a design is created which permits an inner container to be inserted when the spray head is pivoted out.

The spray head itself exhibits a cylindrical, spherical, spiral, wedge, bottle or helical form. The exact form depends on the type and form of the collection device and the liquid-absorbing inserts, as well as the liquids to be drained.

In another preferred embodiment form, the collection device exhibits a lid with at least one inlet, preferably a lid that can be removed and/or pivoted. By this means, both the cleaning and disinfecting of the collection device are facilitated, as is placement of an inner container

As an alternative, the collection device is produced in one material piece including the lid. Here, depending on the selected material, current manufacturing processes are used.

In another embodiment form, a flow sensor and a shutoff valve are placed before the spray head. The object of such a flow sensor is to sound an alarm after a certain filling level has been reached in the collection device and to close a shutoff valve found before the flow sensor or a clamp, so that the collection device does not overflow. In addition, beneath the spray head a pipe section can be attached, which, via an opening in the base, leads out of the collection device and likewise has a shutoff valve available. Behind the pipe section leading out of the collection device, a pump can be attached for generating negative pressure, and be attached via a starter suction line and the shutoff valve with the collection device.

Provision is also preferably made that the device exhibits a comminution device to reduce the size of particulate components of the exudate drawn out by suction.

Further provision is preferably made that the invention-specific device exhibits a drop counter or a weighing device for determining the amount of the wound exudate passing into the collection device. In this way it is ensured that the amount of the drained exudate can be determined exactly which otherwise is not readily possible due to use of the superabsorbing polymers. In a preferred embodiment, the said drop counter is united with the flow sensor.

If a preset range of quantity of transferred exudate is exceeded or fallen short of, provision can be made that the device issues recommendations for increasing or decreasing the suction rate, which the physician then can confirm by pressing a button. If necessary the device itself can even adjust the negative pressure to the indicated range.

Additional provision is preferably made that the invention-specific device additionally exhibits a removal device for removal of fresh exudate. The said removal device can, for example, be configured with flanges and valves customarily used in catheters and infusion equipment. They also can consist of a closable chamber (a polyethylene bottle, for example) that can be pivoted into the liquid stream. In this way, a physician, for example with a commercially available disposable syringe, can remove freshly transferred exudate at any time, and provide it for testing, for example.

In especially preferred fashion, this removal device can be configured so that it takes samples at predetermined time intervals, such as every 15 minutes. These can be analyzed using an analysis device in regard to physiological and/or pathological parameters, and the values obtained can be stored so that they can be read out by a physician at any time.

Alternatively, the samples taken can also preserved for investigation of bacterial load, through fixing or freezing, for example, or be spread on an agar plate. Preferably, provision is made here of a set of agar plates or sample vessels, preferably in the form of a revolving tray, by which time series samples can be taken.

Preferred physiological and/or pathological parameters are

a) pH value of the exudate

b) protein content

c) bacterial load

d) percentage share of blood or blood cells in the transferred liquid, and/or

e) oxygen content.

Here, for example, a pH meter, an oxygen probe, a bacterial detector, a hemoglobin sensor or a protein sensor can be used.

This then can immediately take the necessary measures, for example, administer an antibiotic or topical dose of antimicrobial agents (especially silver, copper, octenidine, antibiotics, etc.) or buffers, or a suitable solution of sodium chloride, ringer or protein solution. If blood or blood cells are present in the transferred liquid, an alarm can be triggered and/or the pumping process interrupted, to prevent acute anemia.

Provision can also be made that the device issues recommendations for wound irrigation, and then carries it out independently, if necessary after confirmation by the physician. For this, via a pump reversal, an irrigation medium can be applied from a container to the wound. The said irrigation solution can especially contain the above-named agents or solutions.

Especially preferred provision is made that the invention-specific device contains a means for determining the bacterial load and/or the material composition of the exudate. Such a device can, for example, be a biosensor (such as a biochip) or a test strip with color indicators.

In regard to the bacterial load, here especially multi-resistant strains are of interest, which, among other things, are responsible for the notorious hospital infections, such as MRSA (methicillin-resistant Staphylococcus aureus), ORSA (Oxacillin-resistant Staphylococcus aureus), VISA (Vancomycin-intermediate Staphylococcus aureus) or VRSA (Vancomycin-resistant Staphylococcus aureus). Such biosensors are based for example on the principle of the immunoassay or that of a biochip—preferably in the form of a disposable, one-time product or on aptamers. Such technologies are known to one skilled in the art from the pertinent literature.

In regard to the material composition of the exudate, especially serum proteins (albumins), matrix metalloproteases, clotting factors and proteins (such as thrombin, fibrin) inflammation markers (cytokines), insulin or glucose are of interest, as well as the markers typical for kidney and liver values. Here also, for example, the immunoassay principle can be realized, preferably in the form of a disposable product (test strips).

Preferably the said biochip, preferably in combination with the operating and/or control elements described below, exhibits a storage device, in which the measured values for bacterial load and/or material composition of the exudate can be inputted, and from which these can again be outputted. In this way, the temporal evolution of the exudate composition can be traced and logged.

Provision is further preferably made that at least one liquid-guiding component of the device exhibits a liquid-repelling (hydrophobic) coating, preferably a lotus effect coating. In this way, moistening of the surfaces is reduced, thus facilitating cleaning and making a long-duration contamination difficult. Especially this can mitigate the consequences of faulty operation—if for example, exudate is drained into a container that, unlike what was planned, has a collection bag, since the requisite cleaning is so much facilitated.

Provision is further preferably made that the invention-specific device exhibits an operating and/or control element comprising a touch screen. The said operating and/or control element can be configured, for example, in the form of a tablet PC. A touch screen has the advantage that it is easier to clean than a traditional keyboard. The data can be transferred between the operating and/or control element and the device, including a pump, via a traditional wire connection, or also wirelessly, for example via a WLAN or Bluetooth or similar protocol. Along with hygienic advantages, a wireless design also has the advantage that the operating and/or control element can be designed to be physically detached from the device.

The operating and/or control element can be configured to be detachable from the rest of the device, particularly detachable from the vacuum pump. In this way, the physician can, for example, take it with him to read out data while the device is in operation on the patient. In addition it is thus possible to design the operating and/or control element as a multi-use product, while the rest of the device or parts thereof, such as the collection device or the vacuum pump, and are configured as disposable, one-time-use products.

Further embodiment forms are made clear in what follows using the illustrations and trials; however, the invention is in no way limited through their specific configuration.

### FIGURES

### List of Reference Symbols

1 wound dressing
2 suction head
3 opening (of 1)
4 drainage hose
5 collection device
6 vacuum pump
7 flow sensor
8 hose connector
9 floor (of 22)
10 housing
11 inlet
12 outlet
13 opening
14 floor
15 inner container
16 liquid-absorbing layer
17 foil or film
18 removal location
19 inner wall
20 starting suction line
21 shutoff valve
22 spray head
23 exit opening
24 three-way valve
25 vacuum line
26 vacuum line
27 return valve
28 storage battery
29 display
30 lid
31 AC attachment
32 pipe connection
33 floor opening
34 shutoff valve
35 DC attachment
36 solar element
37 flow sensor
38 shutoff valve
39 ventilation valve
40 bacterial filter
41 control module
42 LCD display
43 control unit
44 microcamera
45 screen
46 piezoelectric vibration module
47 signal cable
48 comminution device
49 cutter
50 distributor
51 control module
52 pump module
53 touch screen
54 battery compartment
55 pump
56 removable collection device
57 drainage hose
58 attachment belt
59 bellows
60 valve
61 pump
100 drainage device

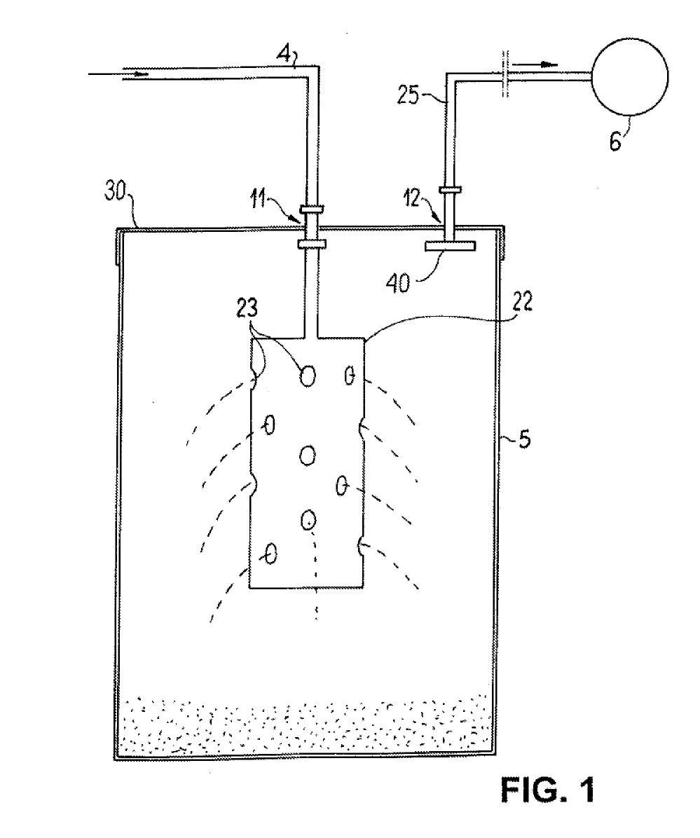

FIG. 1 shows an invention-specific collection device 5 with a lid 30 and spray head 22 with exit openings 23. The drainage hose 4 leads via an inlet 11 to the spray head 22. In the collection device 5, there is a loose filler of liquid-absorbing substances. The drainage device is operated via a vacuum pump 6 and an attached vacuum line 25, which is connected with the collection device 5 via an outlet 12. In addition, a bacteria filter 40 is placed upstream of the suction line.

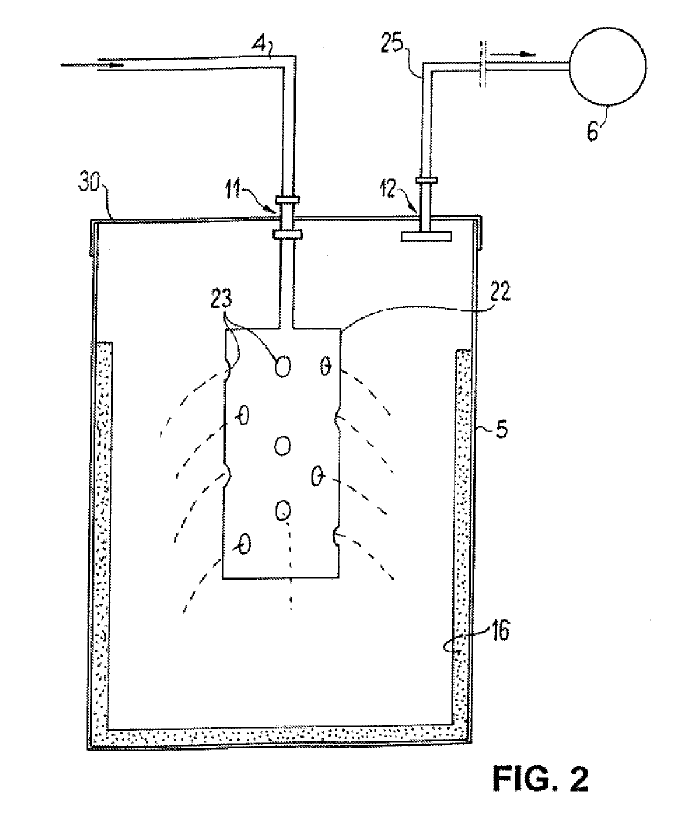

FIG. 2 shows the same design as in FIG. 1, but an inner lining made of an immobilized liquid-absorbing insert 16 is placed in the collection device 5.

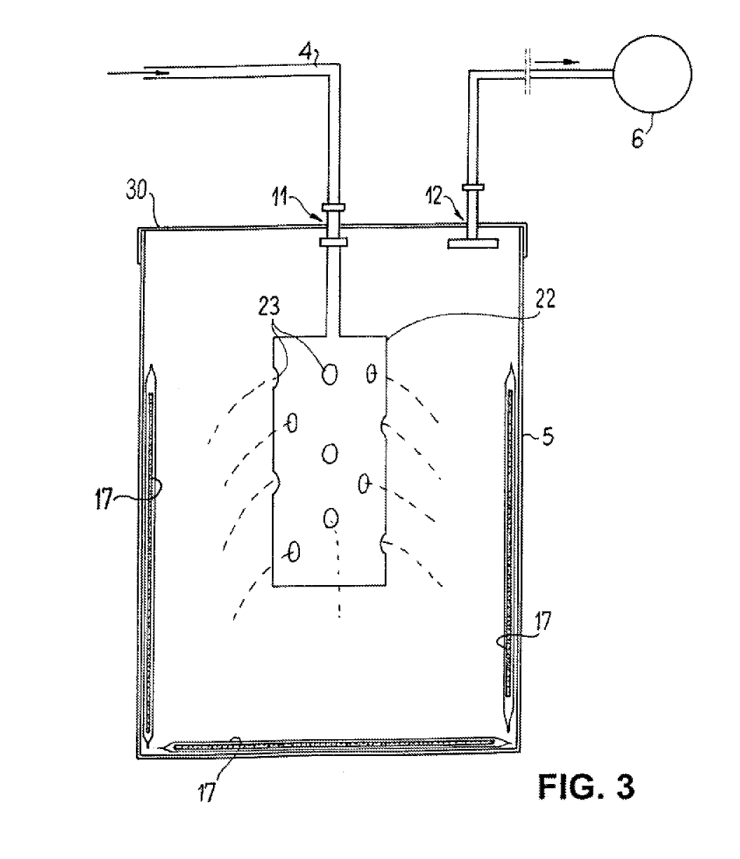

FIG. 3 shows the same design as in FIGS. 1 and 2, but multiple immobilized liquid-absorbing inserts 16, each encased by a foil or film 17, are placed in the collection device 5.

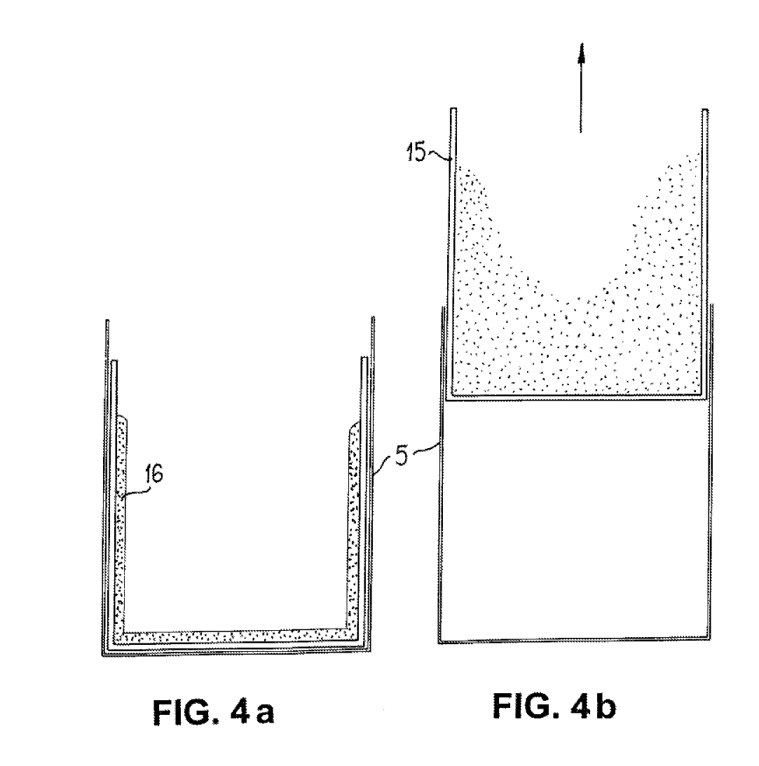

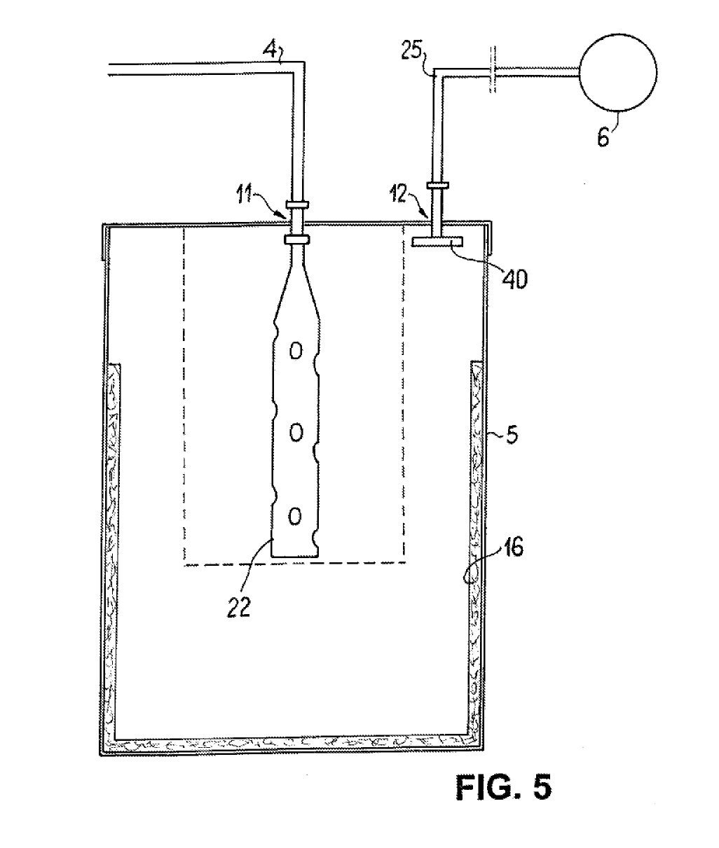

FIG. 4a shows a collection device 5 with an inner container 15 and a liquid-absorbing layer 16.

FIG. 4b shows the removal of the inner container 15 from the collection device 5 with a liquid-absorbing layer 16 in a used state.

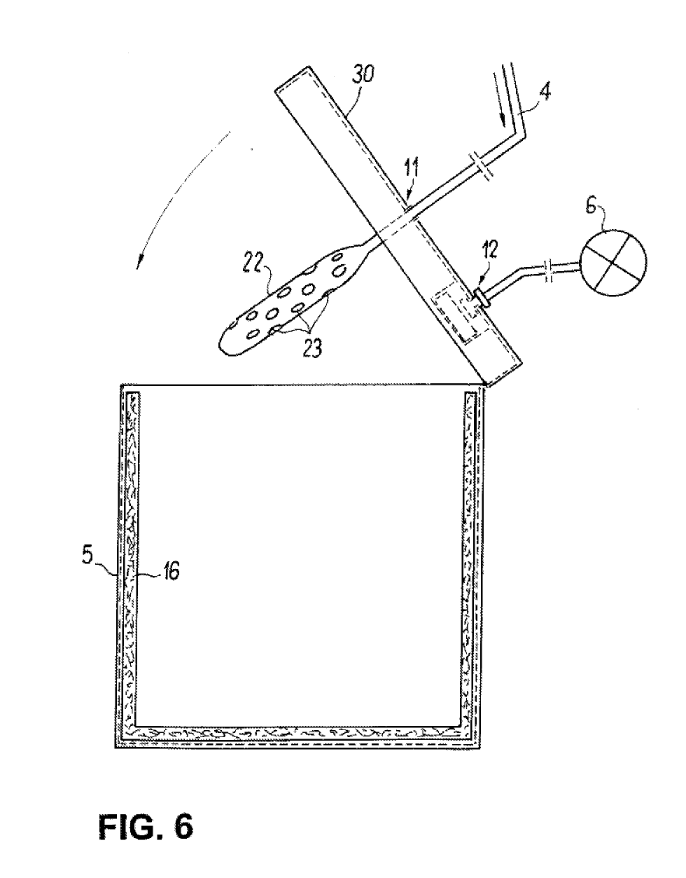

FIG. 5 shows an invention-specific collection device 5 with a lid 30 and a bottle-shaped spray head 22.

FIG. 5 shows an invention-specific collection device 5 with a bottle-shaped spray head 22. The drainage hose 4 leads via an inlet 11 to a spray head 22. In the collection device 5 there is an inner lining of an immobilized liquid-absorbing insert 16. The drainage device is run via a vacuum pump 6 and an attached vacuum line 25, which is connected via an outlet 12 with the collection device 5. In addition, a bacteria filter 40 is placed upstream of the suction line.

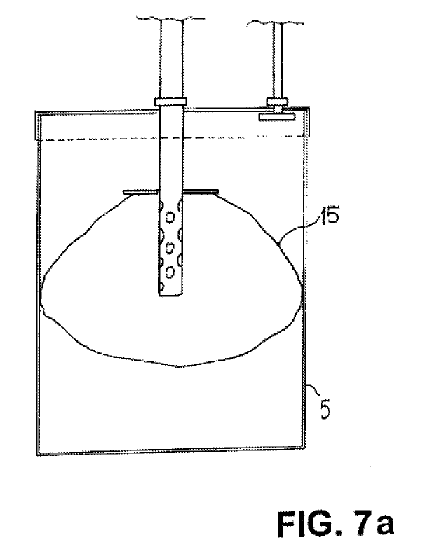

FIG. 6 shows an invention-specific collection device 5 with a pivoting lid 30 and a spray head 22 with an inner lining of an immobilized liquid-absorbing insert 16. In addition, the lid can be locked in an inclined position, to remove the spray head 22 from the lid 30 and to be able to pull out the used inner container from the pot.

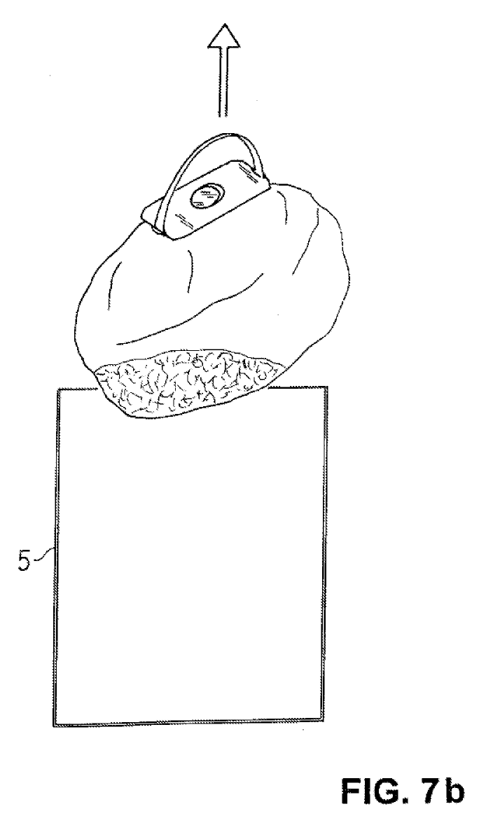

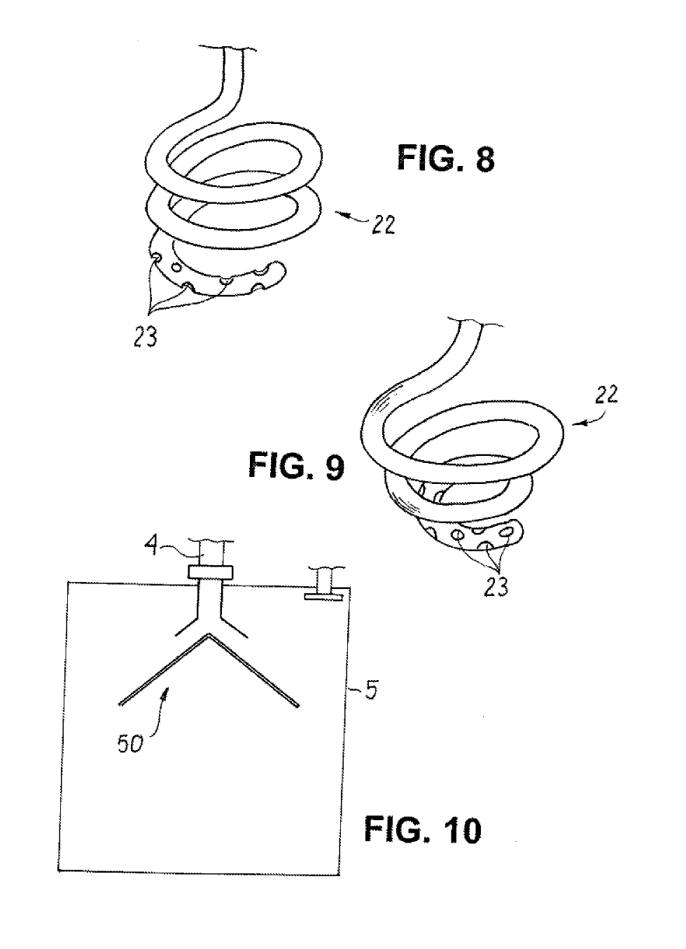

FIG. 7a shows an invention-specific collection device 5 with a bag-shaped inner container 15.

FIG. 7b shows the removal of the bag-shaped inner container 15 from the collection device 5.

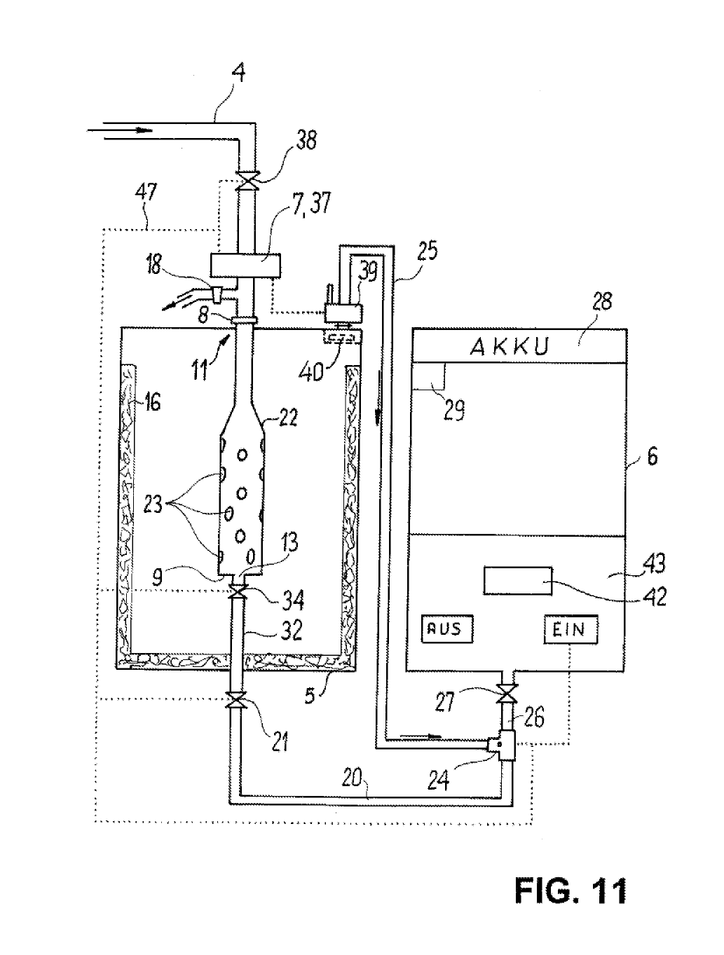

FIG. 8 shows a spiral-shaped spray head 22 with exit openings 23.

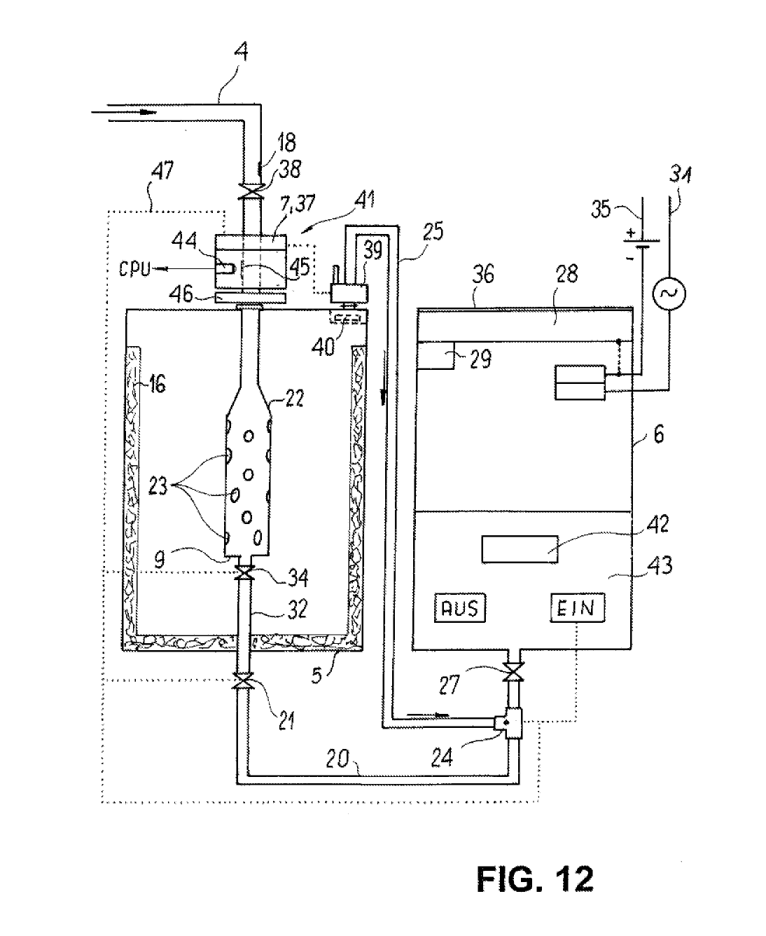

FIG. 9 shows a helical spray head 22 with exit openings 23.

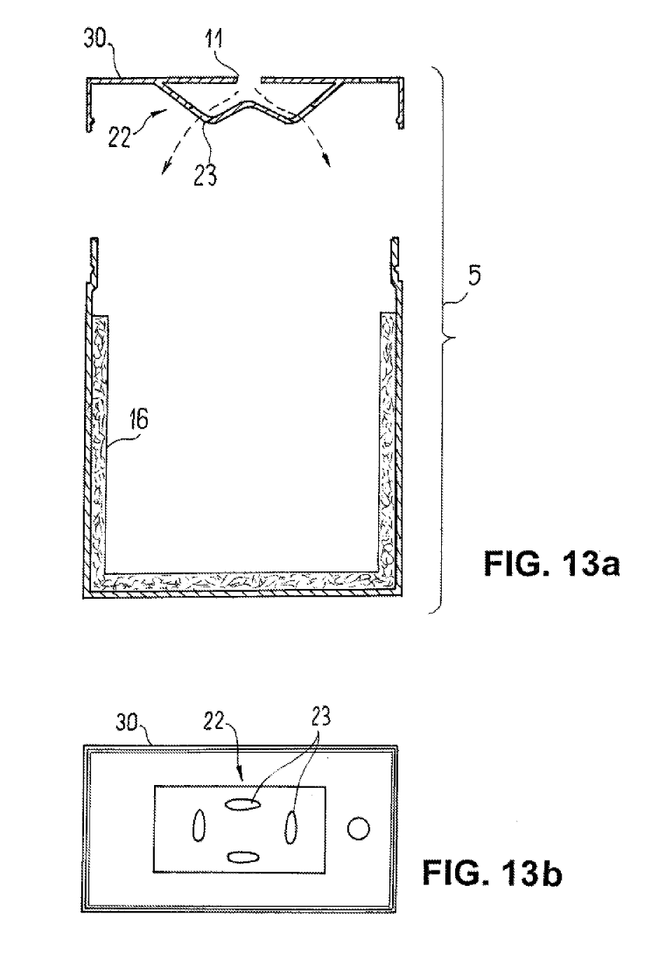

FIG. 10 shows a collection device 5 with a distributor 50 and the discharging drainage hose 4.

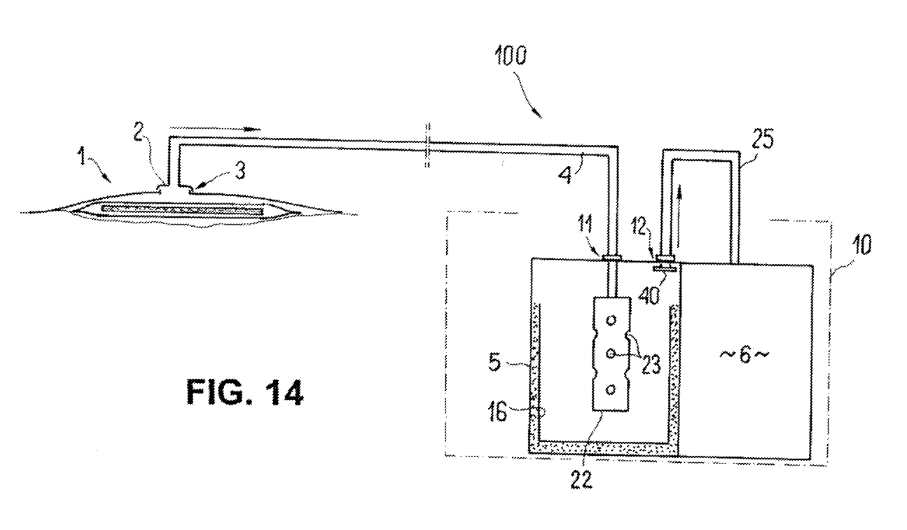

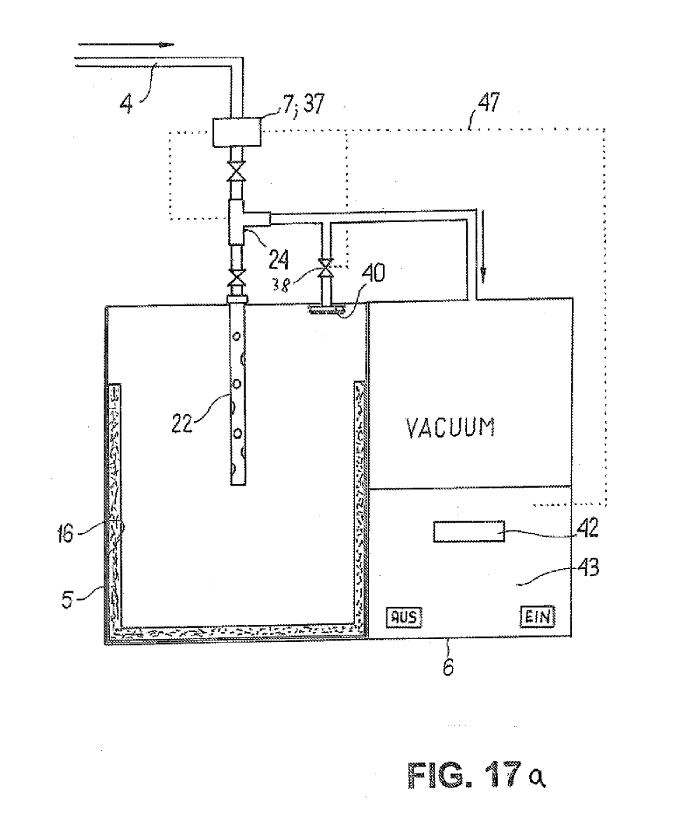

FIG. 11 shows the installed position of the collection device 5 in relation to the entire drainage device 100, depicted in FIG. 14. The drainage hose 4 leads via a flow sensor 7 and a hose connector 8 to the spray head 22. Between the flow sensor 7 and the hose connector 8, a removal location 18 is situated in the form of a sampling valve. The sampling valve can be replaced by an elastomeric membrane (not shown) installed in the drainage hose 4, which constricts after removal of the wound exudate with the aid of a physician's syringe, and seals the hole produced. The spray head 22 exhibits a bottom 9, at which a central opening 13 is situated, to which in turn a pipe section 32 attaches that projects past a bottom 14 of the collection device, protruding outward. Onto the pipe section 32, in the immediate vicinity of the bottom 14, a shutoff valve 34 is installed. The pipe section 32 leads farther via an additional shutoff valve 21 (safety valve) and a suction line 20 to a remote-controlled three-way valve 24, to which vacuum lines 25 and 26 are attached. The one vacuum line 25 connects the collection device 5 with the three-way valve 24 and the other vacuum line 26 connects the vacuum pump 6 with the three-way valve 24. On the vacuum line 26 leading to the vacuum pump 6, optionally there is a check valve 27. The drainage device is run via the control unit 43, with which the vacuum pump 6 and the battery 28 are also connected.

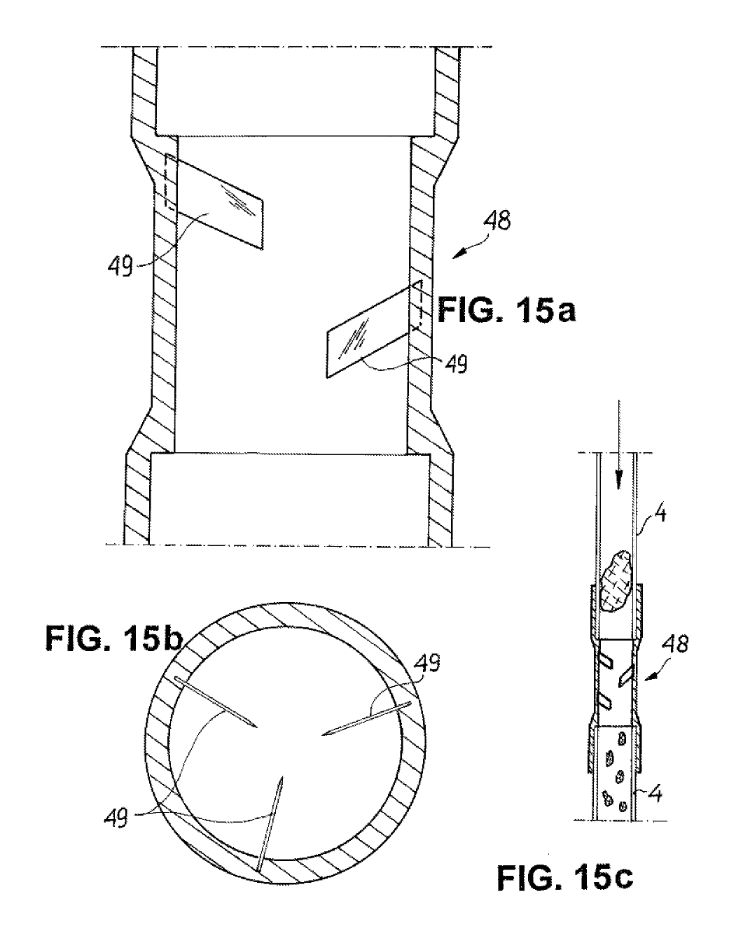

FIG. 12 shows the installed position of the collection device 5 in relation to the entire drainage device 100, depicted in FIG. 14, with various supply units 31 (the alternating current attachment) and 35 (the direct current attachment). By this means, the vacuum pump can be attached as needed to a 230-V alternating-current grid or to a 12-V socket. In addition, a solar element 36 and at least one replaceable, rechargeable battery 28 is provided, the charging status of which can be checked via a display 29 (LED light and/or acoustic signal). Additionally, ahead of the collection device 5 a control module 41 is placed, which is composed of a flow sensor 7, 37 and an optoelectronic element such as a laser transmitter or a microcamera 44, which is aimed at a transparent glass screen 45 placed on the drainage hose 4. The wound exudate pulled out is continuously sampled by the microcamera 44 and analyzed by the control module 41. This in turn makes it possible to assess the healing process based on a comparison with reference data. The changes in the exudate parameters registered by the control module 41 are also received by the control device 43, which can appropriately alter the operating parameters.

The data are passed on to CPU unit (chip), which among other things, has reference data available and which is interlocked with the control unit 43. The control unit 43 is equipped with an LCD display 42. The control unit 43 connected with the control module 41 can provide varied data. Included in these, among others, is the consistency of the wound exudate, the color, cytological information, etc. In addition, the control module 41 can determine whether MRSA, bacteria, etc. are present, and provide appropriate alarms. The vibration module 46 can be used instead of the comminution device 48. Such piezoelectric modules are known and are part of the prior art.

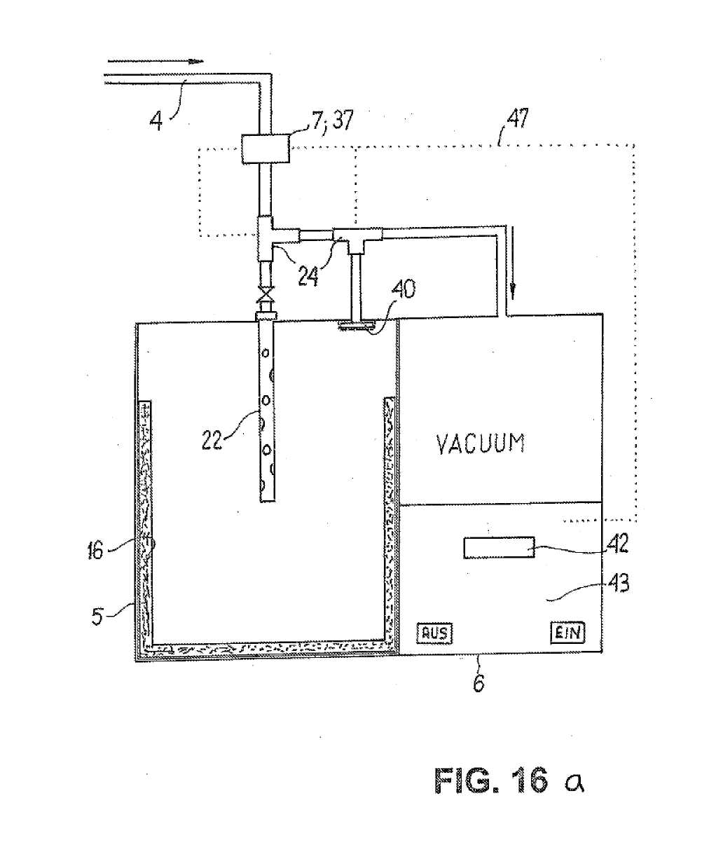

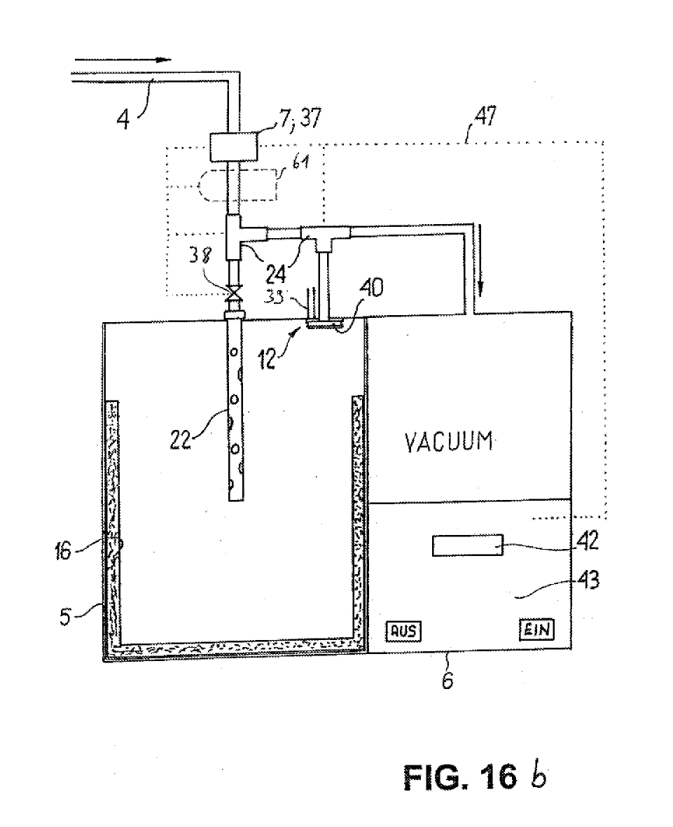

FIG. 13a shows a collection device 5 with a lid 30 and a spray head 22 integrated into the lid.

FIG. 13b shows a top-down view of the lid 30 with the spray head 22 and the exit openings 23.

FIG. 14 shows the invention-specific device 100 in its totality. The wound dressing 1 which possibly comprises an absorption body, is applied directly to the wound on the skin of the patient. While in operation, the device 100 is attached to the electrical grid via the alternating-current connection 36. The shutoff valves 38, 34, 21 and the check valve 27 are open. The vacuum line 25 is closed by the three-way valve 24, as is the ventilation valve 39 on the lid of the collection device. The section from the suction head 2 to the three-way valve 24 is filled with air at atmospheric pressure. By pressing the ON button of the housing of the vacuum pump 6, its drive is started. The air is strongly pulled (about 170 mm Hg) via the start suction line 20, causing the wound exudate to start flowing. The wound exudate gets to flow sensor 7, which emits a signal to a control unit 43, which first closes the shutoff valves 34, 21 and reverses the three-way valve 24, so that the starting suction line 20 can no longer pull in air. The wound exudate flows due to inertia and the existing pressure difference in the spray head 22. In the meantime, the vacuum line 25 is released and the vacuum pump 6 begins to operate at a second, lower stage of about 85 mm Hg. The wound exudate flows via the openings 23 of the spray head 22 into the interior of the collection device 5 and moistens the inner lining 16 found therein, which also contains superabsorber particles. When a certain amount of exudate has been registered by the flow sensor 7, it sends a signal to the control device 43, which in turn closes the shutoff valve 38 situated on the drainage line 4.

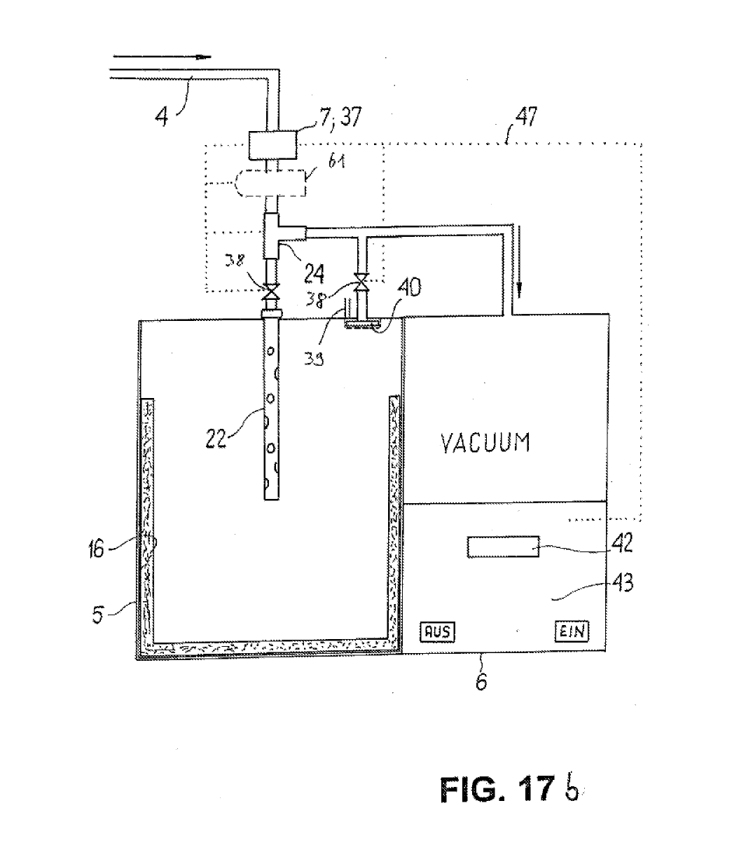

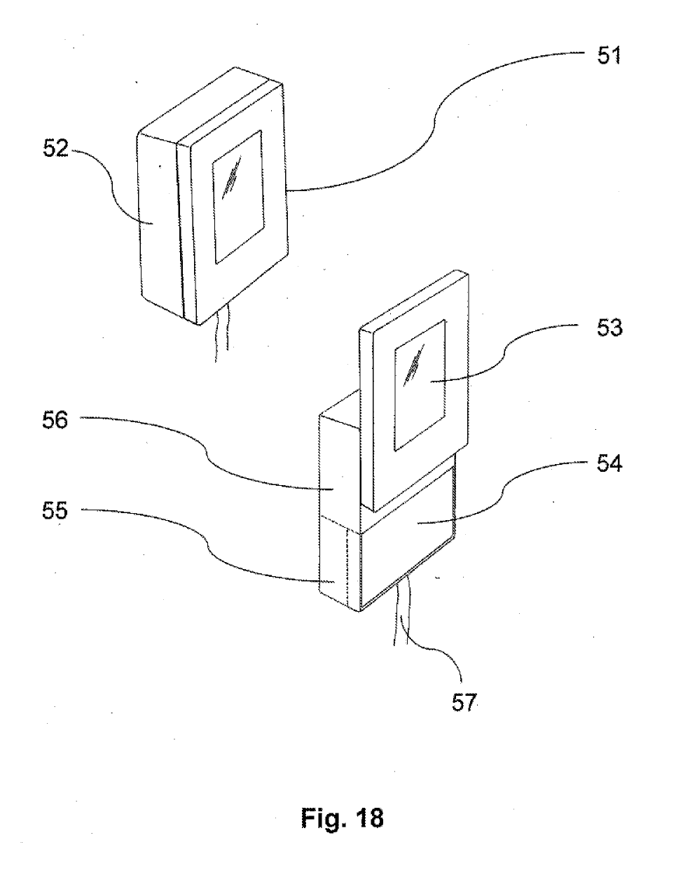

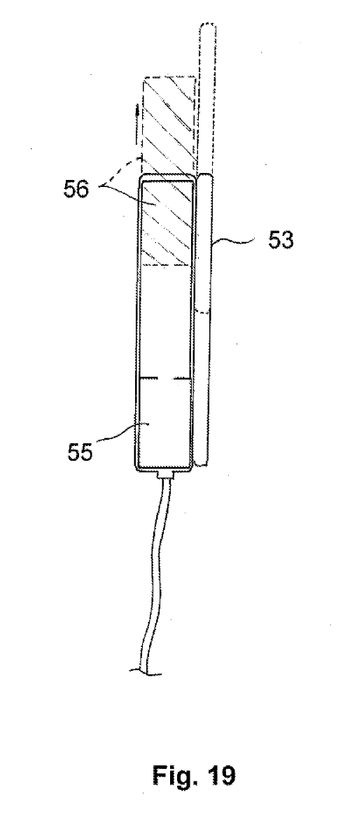

FIG. 15a shows a vertical section of a comminution device 48 for installation in a drainage hose. FIG. 15b shows a comminution device 48 for installation in a drainage hose in cross section. It can be perceived that the cutters 49 project into the lumen of the drainage hose. FIG. 15c shows the installation of a comminution device 48 in a drainage hose 4. It can be perceived that the cutters 49 cause coagulated blood and other particulate components (purulent plaques, biofilm particles, etc.) of the drained exudate to be comminuted.

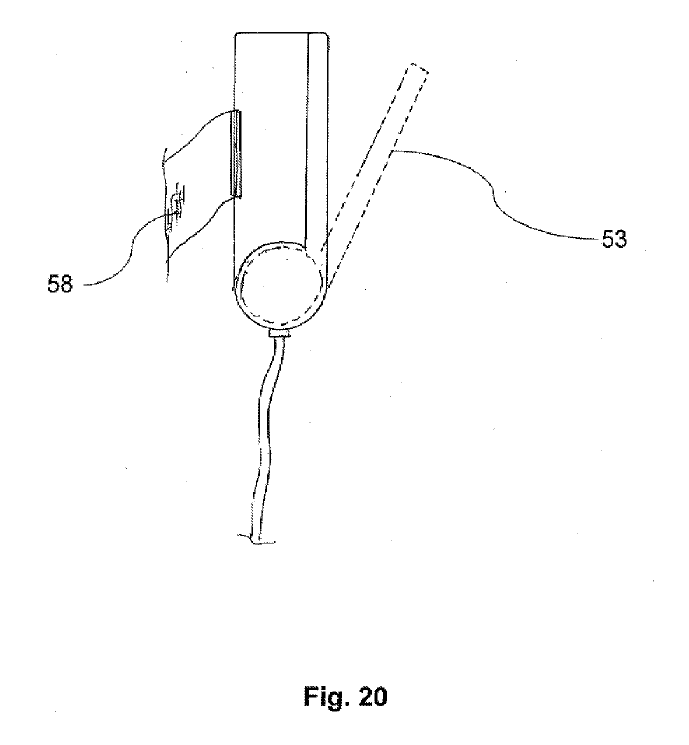

FIGS. 16a-16b and 17a-17b show additional embodiment forms of a stationary vacuum pump. The first three-way valve 24 installed on the drainage hose 4 is set to the “open” position for the vacuum line and in the “closed” position for the spray head 22. The second three-way valve 24 situated on the vacuum line (FIGS. 16a-16b) or the control valve 38 (FIGS. 17a-17b) is likewise closed for the short vacuum line on the lower end of which the bacteria filter 40 is situated. In this setting, the ON button is pressed on the vacuum pump 6. The air is strongly pulled, thus causing the wound exudate to start flowing and getting to the flow sensor 7, which at that moment issues a signal to control unit 43, which resets the first three-way valve 24 into the “open” position and the second three-way valve 24 into the “closed” position for the wound exudate. The function of the vacuum pump 6 is to put the device into motion. Thus, the vacuum pump 6 is a “starter.” In addition, it comprises control unit 43. A smaller pump 61 used on the drainage hose 4 takes on the function of continuous pulling of wound exudate. The pump 61 can also transport smaller air bubbles.

One more vent 39 is provided at the outlet 12 of the collection device 5. The wound exudate flows via the openings 23 in the spray head 22 into the interior of the collection device 5 and moistens the inner lining 16 containing SAP found therein.

When a predetermined amount of exudate has been registered by the flow sensor 7 or by the flow meter 37, it sends a signal to the control unit 43, which in turn closes the shutoff valve 38 found on the drainage hose 4, and simultaneously shuts off the pump 61.

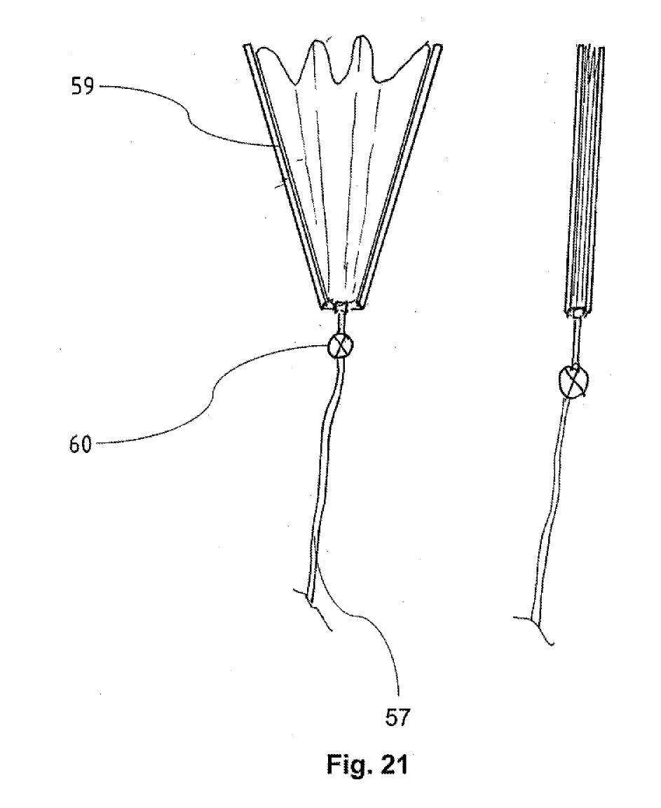

FIG. 18 shows a portable vacuum pump as it can be used in the context of the invention-specific drainage device. The vacuum pump exhibits a control module 51 and a pump module 52. The control module may exhibit a touch screen 53, while the pump module can exhibit a battery compartment 54, a pump 55 and a removable collection device 56. The said collection device can, for example, be clad with superabsorbing polymers. The drainage hose 57 is depicted in truncated fashion. The touch screen can be configured to slide vertically—similar to the sliding function on a cell phone—to make operating elements concealed beneath accessible or to open the battery compartment.

FIG. 19 shows the portable vacuum pump in a side view with the touch screen 53 that can slide vertically and the pump 55. Additionally perceptible is that the collection device 56 is designed so it can be removed upwards. The collection device can be configured via a valve so that upon being inserted it is automatically connected with the drainage hose.

FIG. 20 shows an alternative embodiment form of a portable vacuum pump as it can be used in the context of the invention-specific device. Here the touch screen 53 is in the form of a folding display device—similar to a cell phone with a folded display—to make operating elements concealed beneath accessible or to open the battery compartment. The said collection device can, for example, be clad with superabsorbing polymers. Additionally, the portable vacuum pump exhibits an attachment strap 58, by which the pump can, for example, be attached in an inconspicuous fashion on a patient's leg. For this purpose, the portable vacuum pump can also be configured to also be adapted anatomically to the shape of a patient's leg, for example. The last two points mentioned can also be implemented on the vacuum pump as per FIGS. 18 and 19.

FIG. 21 shows an alternative embodiment form of a portable vacuum pump, which in this case exhibits the form of a bellows 59. The said bellows can for example be clad with superabsorbing polymers. Additionally, this embodiment form exhibits a valve 60, using which a negative pressure can be applied to the drainage hose 57.

### EXAMPLES

### Production of a Liquid-Absorbing Insert for Use in the Invention-Specific Collection Device

Trial 2

A dispersion of carboxymethyl cellulose and boiled and cooled tap water is mixed with a certain amount of glycerine and homogeneously blended. The glycerine additionally acts in a bacteriostatic and fungistatic manner. The solution is uniformly distributed on the bottom of a flat container. Thereafter, an amount of SAP in powder form is strewed on the surface of the solution. The cakelike material thus produced is then baked at a temperature of about 100° C. in a stove for 90 minutes.

After cooling, the surface is treated with droplets of glycerine and water. Then it is heated again to 120° C. for 3 minutes and again cooled to the ambient temperature.

The white-colored planar formed body holds together additional agents like adhesives and is flexible.

Trial 1

The trial was repeated, without a subsequent application of glycerine, but rather with TiO₂. The titanium oxide under certain conditions has a disinfecting effect and in addition is inert. The results of Trials 1 and 2 are in Table 1, below.

TABLE 1

| | |
|---|---|
| Components of trial no. 1: | Components of trial no. 2: |

| | | | |
|---|---|---|---|
| Water | 40 grams | Water | 40 grams |
| SAP | 18 grams | SAP | 18 grams |
| CMC | 2 grams | CMC | 3 grams |
| Glycerine | 10 grams | Glycerine | 18 grams |
| Collagen Powder | 2 grams | TiO₂ | 2 grams |
| | | Collagen Powder | 2 grams |

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A collection device for negative pressure wound therapy, comprising:
a negative pressure source operably coupled to a container, wherein the container includes at least one liquid-absorbing insert containing polymers.

2. The collection device according to claim 1, wherein the at least one liquid-absorbing insert comprises a foam, a textile material, or a homogeneous non-textile polymeric material.

3. The collection device according to claim 2, wherein the liquid-absorbing insert is at least partially surrounded by a liquid-permeable film.

4. The collection device according claim 3, wherein the liquid-absorbing insert and the container includes a bag disposed there between.

5. The collection device according to claim 4, wherein the container is a disposable container.

6. The collection device according to claim 4, wherein the liquid-absorbing insert is connected with an inner wall of the container.

7. The collection device according to claim 6, further comprising a spacer media that is disposed in the container, whereby the spacer media counteracts the expansion of the liquid-absorbing insert toward the middle of the container when exudates are absorbed by the liquid-absorbing insert.

8. The collection device according to claim 7, further comprising at least one spray head attached to an inlet that is operably with the container, wherein the at least one spray head includes a plurality of exit openings operable to dispose exudate into the container.

9. The collection device according to claim 8, wherein the at least one spray head is pivotable or rotatable.

10. The collection device according to claim 9, wherein the at least one spray head includes a shape selected from the group consisting of: a cylinder, sphere, spiral, wedge, bottle, and helix.

11. The collection device according to claim 10, wherein the liquid-absorbing insert includes a maximum expansion capacity in a moistened state that is adapted to the inner volume of the container.

12. An analysis device for investigation of the exudates comprising: a suction system for analyzing physiological and/or psychological parameters of the exudate; and a liquid absorbing insert operably coupled with the suction system.

13. The analysis device according to claim 12, whereby the said suction is generated by application of atmospheric negative pressure or a suction force mediated by superabsorbing polymers in the liquid-absorbing insert.

14. The analysis device according to claim 13, wherein the suction system is operably coupled to an analysis unit selected from the group consisting essentially of: a drop counter or a weighing device to determine the amount of wound exudate drained into the collection device; a removal device for removal of fresh exudate; a pH meter, oxygen probe, bacteria detector, hemoglobin sensor, or protein sensor; a plurality of test strips; a biochip; a microphotometer; and an immunoassay.

15. The analysis device of claim 14, further comprising a wound dressing operably coupled to suction system.

16. A drainage device for negative pressure wound therapy, comprising: a collection device operably coupled to an analysis unit; a gastight wound-covering dressing operably coupled to a suction head; at least one drainage hose operably coupled to the suction head and the collection device; and a source of negative pressure operably coupled to the collection device.

17. The drainage device according to claim 16, wherein said source of negative pressure is selected from the group consisting essentially of: an electrically driven vacuum pump; a manual vacuum pump, and an evacuated vacuum vessel.

18. The drainage device according to claim 17, wherein the analysis unit is selected from the group consisting essentially of: a drop counter or a weighing device to determine the amount of wound exudate drained into the collection device; a removal device for removal of fresh exudate; a pH meter, oxygen probe, bacteria detector, hemoglobin sensor, or protein sensor; a plurality of test strips; a biochip; a microphotometer; and an immunoassay.

19. The drainage device according to claim 18, wherein the collection device further comprising a liquid-absorbing insert contained therein.

20. The drainage device according to claim 19, wherein the liquid-absorbing insert is at least partially surrounded by a liquid-permeable film.
